(19) United States
(12) Patent Application Publication
(10) Pub. No.: US 2024/0366278 A1
(43) Pub. Date: Nov. 7, 2024

(54) DEVICE FOR DISPENSING BONE CEMENT BY MEANS OF SURGICAL ROBOTS

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 18/649,218

(22) Filed: Apr. 29, 2024

(30) Foreign Application Priority Data
May 4, 2023 (EP) 23171441.1

(51) Int. Cl. A61B 17/88; A61B 34/20; A61B 34/32

(52) U.S. Cl. CPC A61B 17/8833; A61B 34/32; A61B 2017/8838; A61B 2034/2055

(57) ABSTRACT

A device for dispensing bone cement by means of a machine, comprising a connection unit configured for connection to a surgical robot, a press-out unit configured for dispensing bone cement, a receiving unit configured for receiving a container containing bone cement, and an interface configured for connection to a control unit, in order to dispense a predefined amount of bone cement from the container by means of the press-out unit.

### CROSS-REFERENCE TO RELATED APPLICATIONS

This application claims priority pursuant to 35 U.S.C. 119 (a) to European Application No. 23171441.1, filed May 4, 2024, which application is incorporated herein by reference in its entirety.

### FIELD OF THE INVENTION

The present invention relates to the field of medical technology, in particular devices for dispensing bone cement by means of surgical robots. The devices can be used during the implantation of medical implants, such as joint spacers. Such a device can be connected to surgical robots or can be part of a surgical robot. Methods for machine application of PMMA bone cement are also described.

### TECHNICAL BACKGROUND

PMMA bone cements are used in surgery and orthopedics for fixing joint endoprostheses in bone tissue. PMMA bone cements are usually composed of a liquid monomer component and a powder component. The monomer component usually contains the monomer methyl methacrylate and an activator (N,N-dimethyl-p-toluidine) dissolved therein. The powder component, also referred to as bone cement powder, usually contains one or more polymers produced on the basis of methyl methacrylate and comonomers, such as styrene, methyl acrylate or similar monomers, by polymerization, preferably suspension polymerization, a radiopaque agent and the initiator dibenzoyl peroxide. When the powder component is mixed with the monomer component, a plastically deformable dough, the actual bone cement, is produced as a result of swelling of the polymers of the powder component in the methyl methacrylate. When the powder component is mixed with the monomer component, the activator N,N-dimethyl-p-toluidine reacts with dibenzoyl peroxide to form radicals. The radicals formed initiate the radical polymerization of the methyl methacrylate. As the polymerization of the methyl methacrylate progresses, the viscosity of the cement dough increases until the cement dough solidifies. The starting components described above, the bone cement dough produced therefrom and the bone cement produced as a result of this bone cement dough hardening are referred to herein both individually and in their entirety as “bone cement”.

Polymethyl methacrylate bone cements can be mixed in suitable mixing beakers with the aid of spatulas by mixing the cement powder with the monomer liquid. Air bubbles may be incorporated in the bone cement dough, which can negatively influence the mechanical properties of the hardened bone cement. The bone cement dough mixed in the mixing beakers is usually applied using spatulas.

In order to avoid air inclusions in the bone cement dough, a plurality of vacuum cementing systems are described in which the cement components are mixed in cartridges using manually operated mixers; the following documents are cited as examples thereof: U.S. Pat. Nos. 6,033,105A, 5,624,184A, 4,671,263A, 4,973,168A, 5,100,241A, WO99/67015A1, EP1020167A2, U.S. Pat. No. 5,586,821A, EP1016452A2, DE3640279A1, WO94/26403A1, EP1005901A2, U.S. Pat. No. 5,344,232A.

A further development in cementing technology are cementing systems in which both the cement powder and the monomer liquid are already packaged in separate compartments of the cartridge systems and are mixed with one another in the cartridge system only immediately before the cement application. Such closed “full pre-packed mixing systems” are described in EP0692229, DE102009031178, U.S. Pat. Nos. 5,997,544, 6,709,149, DE69812726 and U.S. Pat. No. 5,588,745.

A full pre-packed mixing system was described in the patent specifications DE102016121606B4 and DE102016121607B4. In this mixing system, the cartridge is held vertically with the cartridge head vertically at the top and the monomer liquid is pressed into compressed cement powder from below by means of a press-out device, the cement powder being wetted by the monomer liquid and the air located between the cement powder particles being pressed out and up by the monomer liquid. A bubble-free cement dough is produced without the action of mechanical mixing devices. As is presented in more detail below, such full pre-packed mixing systems can be used particularly advantageously together with the devices described herein.

### Preferred Embodiments

In the case of the vacuum mixing systems known to date and pre-packed mixing systems, the PMMA cement dough is often applied such that a cartridge filled with PMMA cement dough is connected to a manually operable press-out device, and subsequently the cement dough is manually pressed out of the cartridge by means of this press-out device. This can take place, for example, within the process of the implantation of a knee joint endoprosthesis. As the cement is being pressed out, the medical user can move the dispensing tube over the surfaces of the knee joint endoprosthesis which are to be cemented and, if necessary, over the osseous implant bed of the tibia and of the femur. The cement dough can thereby be distributed uniformly, and the tibial and femoral components of the knee joint endoprosthesis can subsequently be pressed onto the previously prepared tibia and the prepared femur. Uniform cement distribution and a homogeneous bond between the bone tissue and the joint components can thereby be made possible. The excess cement dough can be removed manually. When cementing hip joint endoprostheses, the acetabular cup can be cemented in a similar way. For cementing the shaft of hip joint endoprostheses, the previously prepared channel in the proximal femur is retrogradely filled with cement dough. The cement dough is then pressed into the spongy bone by means of a pressurizer. The shaft of the hip prosthesis is then inserted into the channel of the femur filled with bone cement dough, thereby displacing the excess bone cement dough.

Surgical robots are increasingly used in surgery and orthopedics. They play a role in particular in the implantation of cement-free knee joint endoprostheses. It would be desirable if, for example, cemented prostheses, such as knee joint and hip joint endoprostheses, could also be implanted with the aid of surgical robots.

One object of the invention is to provide a device for machine application of bone cement, for example PMMA bone cement, said device being connectable or connected to a surgical robot. The device can make possible the machine application of bone cement volumes from cartridges in a predefined time period, said bone cement volumes having been defined by the surgeon beforehand. During the cementing process, the device can preferably be moved by a robot arm.

The device can preferably be able to precisely meter the cement volumes to be pressed out. This metering process can preferably be temporally controllable so that a surgical robot can position the bone cement dough exactly on the implant and on the bone tissue by defined movement in the directions of the x-axis, the y-axis, and the z-axis. For this purpose, the device can be designed to be connected to cartridges which already contain pre-mixed PMMA cement dough. This means that the medical user mixes the cement components in a cartridge to form a PMMA bone cement dough and subsequently connects the cartridge to the device, which is already located on the robot arm or can be connected to the robot arm. In some embodiments, the device can be connected to a cartridge which contains the unmixed cement components, for example a monomer liquid and a cement powder. By means of a device as described herein, in this case the cement components can be mixed by a movement of the dispensing plunger toward the cartridge head according to patent specifications DE102016121606B4 and DE102016121607B4 and subsequently pressed out of the cartridge after formation of the bone cement dough. In one variant, the device described herein can be designed such that the use of electric motors and complex transmissions for pressing out the bone cement dough is unnecessary. For example, pneumatic or hydraulic drives can be provided for this purpose. The source of the pneumatic or hydraulic pressure can preferably be arranged outside the device described herein and outside the connected robot. The weight load of the robot arms can thereby be limited, and cost-effective production of the device can be possible. In some embodiments, the device described herein can be configured to carry out the process of pressing out the bone cement using electric motors and transmissions. Furthermore, in some embodiments, well-controllable energy sources already available in the operating theater for driving purposes can be used for driving the device described herein.

An object of the present invention is to solve one or more of the problems described above and further problems of the prior art. For example, the invention enables dispensing of bone cement by means of a machine, in particular by means of a surgical robot.

These objects are achieved by the methods and devices described herein, in particular those which are described in the claims.

Preferred embodiments of the invention are described below.

A first embodiment describes a device for dispensing bone cement by means of a machine, comprising a connection unit configured for connection to a surgical robot, a press-out unit configured for dispensing bone cement, a receiving unit configured for receiving a container containing bone cement, and an interface configured for connection to a control unit, in order to dispense a predefined amount of bone cement from the container by means of the press-out unit.

A second embodiment describes a device according to the first embodiment, wherein the interface is configured to convey the amount of bone cement dispensed by means of the press-out unit and to stop further dispensing of the bone cement when the predefined amount is reached.

A third embodiment describes a device according to any one of the preceding embodiments, which also comprises a sensor operatively connected to the interface, in order to determine the amount of bone cement dispensed by means of the press-out unit.

A fourth embodiment describes a device according to the third embodiment, wherein the sensor is an optical sensor, a mechanical sensor or a magnetic sensor.

A fifth embodiment describes a device according to the third or fourth embodiment, wherein the sensor is configured to detect a spatial position of a marking in order to thereby determine the amount of bone cement dispensed by means of the press-out unit, the marking preferably being a colored component of the press-out unit, particularly preferably a sealing ring of a plunger.

A sixth embodiment describes a device according to any one of the third to fifth embodiments, wherein the sensor and/or the interface is/are configured to determine an inner volume of the press-out unit and/or of the container.

A seventh embodiment describes a device according to any one of the third to sixth embodiments, wherein the sensor is arranged on a sensor strip which extends along the receiving unit on the outside of the device.

An eighth embodiment describes a device according to any one of the preceding embodiments, wherein the connection unit comprises an electrical connection point which is configured to supply power to the device and/or to receive a control signal.

A ninth embodiment describes a device according to any one of the preceding embodiments, wherein the connection unit comprises a connection point for connection to a pressurized medium, the pressurized medium preferably being compressed air, a vacuum source or a hydraulic medium.

A tenth embodiment describes a device according to any one of the preceding embodiments, wherein the press-out unit comprises a drive configured for dispensing bone cement by means of the press-out unit.

An eleventh embodiment describes a device according to the tenth embodiment, wherein the drive is a hydraulic, pneumatic or electric drive.

A twelfth embodiment describes a device according to the tenth or eleventh embodiment, wherein the interface is configured to control the drive.

A thirteenth embodiment describes a device according to any one of the preceding embodiments, the device also being configured for mixing bone cement by means of a machine in the container.

A further aspect relates to a computer program for controlling the device according to any one of the preceding embodiments, the computer program being configured to operate the press-out unit and/or the sensor.

A further aspect relates to a kit, comprising a device according to any one of the first to thirteenth embodiments and a container containing bone cement, wherein the container is preferably configured for mixing bone cement.

### DETAILED DESCRIPTION

With respect to the embodiments described herein, the elements of which “have,” or “comprise,” a particular feature (for example, a material), in principle, a further embodiment is always contemplated in which the relevant element consists solely of the feature, i.e., does not comprise any other constituents. The word “comprise” or “comprising” is used herein synonymously with the word “have” or “having.”

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, it is contemplated in principle that all features described herein in connection with a method are also applicable to the products and devices described herein, and vice versa. All such considered combinations are not explicitly listed in all instances, simply in order to keep the description brief. Technical solutions known to be equivalent to the features described herein are also intended in principle to be encompassed by the scope of the invention.

A first aspect of the invention relates, in a first embodiment, to a device for dispensing bone cement by means of a machine, comprising a connection unit configured for connection to a surgical robot, a press-out unit configured for dispensing bone cement, a receiving unit configured for receiving a container containing bone cement, and an interface configured for connection to a control unit, in order to dispense a predefined amount of bone cement from the container by means of the press-out unit.

The devices described herein are provided for dispensing bone cement by means of a machine. Accordingly, these devices are preferably configured to dispense bone cement by means of an electronically controlled drive. The devices can advantageously be used in conjunction with surgical robots. An example of a surgical robot is described in EP3743004A1. Such robots preferably comprise a drive unit and a control unit in order to enable the operation of the robot. The devices described herein comprise an interface configured to be connected to such a control unit of a robot. The device according to the invention can thereby operatively interact with the control unit of the robot. For example, the control unit of the robot can control the dispensing of bone cement from the device according to the invention by means of the interface. For this purpose, the interface can be operatively connected to a press-out unit of the device. In some embodiments, the interface is configured to forward a control signal from the control unit of a robot to the press-out unit. In some embodiments, the interface can be configured to process such a control signal from the control unit and to deliver the processed control signal to the press-out unit.

In some embodiments, the device can be attached to a robot arm of a surgical robot. This can make it possible to control the spatial position of the bone cement dispensed from the device by the robot arm interacting with the device. Three-dimensionally controllable dispensing of bone cement can thereby preferably be carried out.

The starting components of a bone cement which are described herein, for example a powder component and a liquid component, the bone cement dough produced therefrom and the bone cement produced as a result of this bone cement dough hardening are referred to herein both individually and in their entirety as “bone cement”. A preferred example of a bone cement is a PMMA bone cement. The device can be configured either to receive a container which contains fully mixed bone cement or to receive a container which contains a plurality of separated components of a bone cement. The device can be configured to mix these components. This mixing process can preferably be controllable by means of a surgical robot.

The device according to the invention comprises a receiving unit configured for receiving a container containing bone cement. In some embodiments, the receiving unit is configured to receive commercially available dispensing devices or devices for mixing bone cement. In some embodiments, the receiving unit is configured to receive a container which contains the necessary components for producing a bone cement in separate compartments. In one embodiment, the receiving unit is configured to receive a container in which bone cement can be mixed and subsequently dispensed. In some embodiments, the device is configured to mix, by means of a robot, bone cement in a container to be received and to dispense said bone cement by means of the press-out unit. Particularly preferred containers with which the device according to the invention can be used are described in DE102016121606B4 and DE102016121607B4, which are hereby fully incorporated by reference. DE102016121606B4 describes a bone cement applicator for mixing and applying a bone cement, wherein the starting components of the bone cement can be mixed in a closed cartridge to form a bone cement dough, wherein the cartridge comprises a multipart closure system having a dispensing opening, wherein at least two parts of the closure system are movable relative to one another, driven by a movement of the mixed bone cement dough, and the dispensing opening opens as a result of this movement of the at least two parts of the closure system relative to one another, and wherein the movement of the mixed bone cement dough is to be driven by a pressure on the bone cement dough.

To receive a container containing bone cement, the press-out unit can be provided with a suitable connection element which is configured, for example, for form-fitting connection to the container. For example, such a connection element can be a bayonet closure or a snap closure. The connection element can comprise hooks, tabs or threads which enable a form-fitting connection to the container.

The device comprises a press-out unit configured for dispensing bone cement. The press-out unit can comprise, for example, a plunger configured to exert pressure on a received container containing bone cement. The press-out unit can comprise a movable plate configured to exert pressure on a received container containing bone cement. The plunger or plate can be arranged to move axially into a received container containing bone cement.

The device can comprise a housing which, for example, can have a substantially cylindrical basic shape. The press-out unit can be arranged within this housing. An abutment for the press-out unit can be arranged and fastened on the housing. The abutment can be fastened to the housing by means of a screw or rivet, for example.

In one embodiment, the device is configured to determine the amount of bone cement dispensed by means of the press-out unit. This can be done, for example, by determining the position of an element of the press-out unit, for example the position of a plunger or plate which exerts pressure on the container containing bone cement in order to dispense bone cement from the container. For this purpose, the element of the press-out unit can comprise a marking whose position can be determined by means of a sensor.

In one embodiment, the interface is configured to convey to the robot the amount of bone cement dispensed by means of the press-out unit and to stop any further dispensing of the bone cement when the predefined amount is reached.

In one embodiment, the device comprises a sensor. The sensor is preferably operatively connected to the interface in order to determine the amount of bone cement dispensed by means of the press-out unit and/or to convey said amount to the robot.

The sensor can be an optical sensor, a mechanical sensor or a magnetic sensor. The optical sensor can be, for example, a camera or a photodiode. The device can also comprise a light source, for example a light-emitting diode or a laser. The sensor can be, for example, a sensor with propagation time measurement, a sensor with phase modulation, a triangulation sensor, or an interferometric sensor.

The magnetic sensor can, for example, be configured to determine a relative position of a movable part of the press-out unit according to the functional principle of magnetostriction. Such a sensor can contain, for example, a measurement base arranged immovably within the device, a waveguide, a movable magnet, and a signal converter. Mechanical vibration can be generated in the waveguide by a magnetic force. By determining the propagation time of this vibration, distances between two defined points can be measured. The signal converter can be configured to convert a mechanical vibration of the sensor into a measurement signal.

The sensor can be configured to detect the travel distance of an element of the press-out unit. For example, this can be accomplished by the sensor operatively interacting with a drive of the press-out unit.

In one embodiment, the sensor is configured to detect a spatial position of a marking in order thereby to determine the amount of bone cement dispensed by means of the press-out unit. The marking can preferably be a colored component of the press-out unit, for example a sealing ring of a plunger or plate. The marking can differ in color from the elements of the device that surround the marking.

In one embodiment, the sensor is configured to determine an inner volume of the press-out unit. In one embodiment, the sensor is configured to determine an inner volume of the container containing bone cement. In one embodiment, the interface is configured to determine an inner volume of the press-out unit. In one embodiment, the interface is configured to determine an inner volume of the container containing bone cement.

In this way, the amount of bone cement dispensed by means of the device can be determined.

The sensor can be arranged on a sensor strip. The sensor strip can expediently be attached to the device in order to determine the amount of bone cement dispensed by means of the press-out unit. In one embodiment, the sensor strip extends on the outside of the device along the receiving unit or along the press-out unit. The sensor strip can extend along the device in the axial or longitudinal direction. If necessary, a plurality of sensors can be provided. The sensors can, for example, be arranged peripherally along the outside of the housing of the device.

In a further embodiment, the connection unit comprises an electrical connection point. The electrical connection point can be configured to supply power to the device. In particular, the electrical connection point can be configured to supply power to the press-out unit and/or to the sensor. Alternatively or additionally, the electrical connection point can be configured to receive a control signal. Such control signals can be used, for example, for external control of the different parts of the device by a surgical robot or by another external control device. For example, the sensor and/or the press-out unit can be externally controlled and/or monitored in this way.

In one embodiment, the connection unit has a connection point for connection to a pressurized medium. The pressurized medium can be, for example, compressed air, a vacuum source or a hydraulic medium. The pressurized medium has a pressure which is significantly higher or significantly lower than the usual ambient pressure of approximately 1000 hPa. The pressurized medium can be used to drive the press-out unit. Accordingly, in one embodiment the press-out unit is configured to be driven by a pressurized medium.

In one embodiment, the press-out unit comprises a drive which is configured for the dispensing of bone cement by means of the press-out unit. For this purpose, the drive can be operatively connected to the press-out unit so that the press-out unit can effect the dispensing of bone cement by acting on a container containing bone cement.

In one embodiment, the drive is a hydraulic, pneumatic or electric drive. A hydraulic or pneumatic drive can be fluidically connected to a connection point as described above for connection to a pressurized medium. The device can accordingly comprise an inlet and an outlet for a pressurized medium. The inlet and/or the outlet can comprise a mechanical or electrical controller. The inlet and/or the outlet can each comprise a valve. In particular if the device comprises a pneumatic drive, the outlet can also comprise a filter in order to clean the discharged fluid medium, for example compressed air. Filters that can prevent contamination of the ambient air with germs are particularly advantageous. Such filters are also referred to in the field as sterile filters. Such filters can have, for example, a pore size of 50 to 500 nm, the pore size relating to the nominal volume of the retained particles.

In one embodiment, the interface is configured for controlling the drive. For example, the interface can forward an external control signal to the device in order to control an electric motor or a control valve of a hydraulic or pneumatic drive so as to dispense a predefined amount of bone cement by means of the device.

The device comprises a receiving unit configured to receive a container containing bone cement. The container can either contain fully mixed bone cement or can separately contain individual components of a bone cement. In one embodiment, the device is configured for machine mixing of bone cement in the container. In this case, the bone cement does not have to be mixed by the operating theater personnel, but rather the device takes on the mixing process, possibly under the control of a surgical robot which accordingly controls the device. According to such an embodiment, the bone cement can thus be both mixed and dispensed by a surgical robot with the aid of the device. In one embodiment, the device is configured to dispense the bone cement in a targeted manner in one or more predefined spatial positions by means of a surgical robot. This can render manual guidance of a bone cement dispensing device unnecessary for the surgeon performing the treatment, since this task is taken on by the surgical robot with the aid of the device described herein.

A further aspect relates to a computer program for controlling the device described herein. The computer program can in particular be configured to operate the press-out unit. Alternatively or additionally, the computer program can be configured to operate the sensor. The computer program can be stored on a data carrier. Accordingly, one embodiment also comprises a data carrier on which such a computer program is stored. The computer program can, for example, comprise one or more or all of the following steps, or be configured to carry out these steps:
1) activating the press-out unit, wherein the dispensing of bone cement by means of the device occurs as a result of activating the press-out unit;
2) calculating the amount of bone cement dispensed by means of the device, preferably on the basis of a measured value from a sensor of the device;
3) comparing this amount with a predefined value;
4) deactivating the press-out unit if the amount reaches or exceeds the predefined value.

A further aspect relates to a kit, comprising a device as described herein and a container containing bone cement, wherein the container is preferably configured for mixing and/or dispensing bone cement.

The container can be a full pre-packed mixing system. Full pre-packed mixing systems are cementing systems in which both the cement powder and the monomer liquid are packaged in separate compartments of the cartridge systems in order to be mixed with one another in the cartridge system to form bone cement only immediately before the cement application.

Examples of full pre-packed mixing systems are described in DE102016121606B4 and DE102016121607B4. In such a mixing system, the cartridge can be held vertically with the cartridge head vertically at the top and the monomer liquid can be pressed into compressed cement powder from below by means of a press-out device, wherein the cement powder can be wetted by the monomer liquid, and the air located between the cement powder particles can be pressed out and up by the monomer liquid.

The container preferably contains a connection element, for example a form-fitting connection element, which is configured for connection to a device as described herein. The container can also comprise a mixing rod. The kit can also comprise a nozzle for discharging bone cement from the container. The bone cement can contain an antibiotic.

A further aspect of the invention is a surgical robot which is configured to dispense bone cement by means of a device as described herein, a computer program as described herein and/or a kit as described herein. The surgical robot can contain a device as described herein as an integral part or can merely be configured for connection to such a device. For example, the surgical robot can comprise connection points for the connection element of the device. The robot can be configured to monitor the sensor of the device and/or to control the press-out device of the device. The robot can be configured for operative connection to an electrical connection point of the device as described herein. The robot can also comprise connection points for supplying the device with a pressurized medium, which can be used to drive the press-out device. The surgical robot can be designed and configured for mixing bone cement and/or for dispensing bone cement by means of a device as described herein.

The surgical robot can also contain a computer program for controlling a device as described herein. In this regard, reference is made to the previous statements herein.

A further aspect of the invention is a therapeutic treatment method comprising dispensing bone cement by means of a device as described herein, a computer program as described herein, a surgical robot as described herein and/or a kit as described herein.

### Examples

The invention is further illustrated below using examples which are, however, not to be understood as limiting. It will be apparent to a person skilled in the art that other equivalent means may be used similarly in place of the features described here.

### FIGURES

FIG. 1 shows, by way of example, an embodiment of a device 100 according to the invention. The device 100 comprises a connection unit 101 configured for connection to a surgical robot 200. In the example shown here, the connection unit 101 also comprises an inlet 110 and an outlet 111 which are each used for connection to an external pressurized medium. For example, a compressed-air source, which can be used to drive the press-out unit 102 of the device, can be connected to the inlet 110. The compressed air used is subsequently led out of the device again via the outlet 111. The outlet 111 can comprise a filter in order to clean the air exiting therefrom. The connection unit 101 also comprises an interface 105 configured for controlling the press-out unit 102. For example, a surgical robot operatively connected to the device 100 by means of the connection unit 101 can control, via the interface 105, the amount of bone cement dispensed by means of the press-out unit 102. The device also comprises a sensor 106 in order to detect the amount of bone cement dispensed by means of the press-out unit 102. The sensor 106 is connected, via the interface 105, to an external controller which can be part of a surgical robot 200. The press-out unit 102 is moved by means of a drive 109 in order to dispense bone cement from the device 100. The device also contains a receiving unit 103 configured to receive a container 104 containing bone cement. For this purpose, the receiving unit can comprise a form-fitting connection element, such as a bayonet closure or a thread.

FIG. 2 shows a cross-sectional view of a device according to the invention which is connected, by means of the receiving unit 103, to a container 104 containing bone cement. The bone cement is arranged in an inner volume 108 of the container 104. The press-out unit 102 comprises a plate 112, which is movably arranged within the container 104 and is configured to discharge the bone cement from the inner volume 108. The position of the plate 112 is monitored by means of a sensor 106, whereby the amount of bone cement discharged can be determined.

FIG. 3 shows a further embodiment of a device 100 according to the invention. A commercially available cartridge 104 containing bone cement is attached to the receiving unit 103 of the device. The device comprises a sensor 106, which in this example is designed as a sensor strip comprising a plurality of individual sensor elements arranged in a row.

FIG. 4 shows a device 100 according to the invention in accordance with FIG. 3, the device being connected to a robot 200 by means of a connection unit 101. The sensor 106 can be read by means of the robot 200, since the sensor is operatively connected to the robot by means of the connection unit 101.

FIG. 5 shows a further embodiment of a device 100 according to the invention, the device comprising, in this example, an electric motor as a drive. The device 100 is connected to a cartridge 104 which contains bone cement and which, in this example, also comprises a removable mixing rod allowing manual mixing of multi-component bone cement. The bone cement is arranged in an inner volume 108. The device comprises a press-out unit 102 comprising a plate 112 for discharging the bone cement.

FIG. 6 shows a further embodiment of a device 100 according to the invention, the device comprising, in this example, a compressed air connection point as a drive, which comprises an inlet 110 and an outlet 111. By means of the drive, the press-out unit 102 can dispense the bone cement from the inner volume 108 of the container 104 to the outside, for which purpose the container also comprises a nozzle for discharging the bone cement.

FIG. 7 shows a further embodiment of a device 100 according to the invention, said device being configured for machine mixing of a liquid component and a solid component of a multi-component bone cement. Here, the device 100 is connected to a container 104 which comprises a chamber 113 for holding a liquid component and a chamber 115 for holding a powder component. In this example, the liquid component is arranged in a plurality of film bags 114 in the chamber 113. Such a container is also referred to here as a “full pre-packed mixing system”.

FIG. 8 shows the process of mixing the two bone cement components in a device 100 according to FIG. 7. The press-out unit has a plurality of pins in order to pierce the film bags 114 which contain the liquid component. By exerting pressure on the chamber 113, the film bags are first pierced, and the exiting liquid is pushed out of the chamber 113 and into the chamber 115 for the powder component so that the two components of the bone cement can mix and can bond to one another. The mixing process described here can be controlled by a surgical robot. In the embodiment shown here, the drive unit comprises an electric motor.

FIG. 9 shows a further embodiment of a device 100 according to the invention, said device being configured for machine mixing a liquid component and a solid component of a multi-component bone cement. In this example, the device is equipped with a connection point for a pressurized medium, such as compressed air. The bone cement which has been machine-mixed by means of the device can be discharged from the inner volume 108 by means of a nozzle. The amount of bone cement discharged is monitored by means of a sensor strip. A robot 200 operatively connected to the device 100 controls the press-out unit 102 on the basis of a signal from the sensor 106 in order to dispense a predefined amount of bone cement.

FIG. 10 shows parts of a kit, comprising a device 100 according to the invention which has a connection unit 101, a receiving unit 103 and a sensor. The kit also contains a container 104 containing bone cement, which container can be connected to the device 100 by means of the receiving unit 103. The kit optionally also contains a nozzle 116 for discharging bone cement, which nozzle can be attached to the container 104.

FIG. 11 shows a cross section through one embodiment of a device 100 according to the invention in which the press-out unit 102 comprises a marking 107 which, in the embodiment shown here, is designed as a sealing ring of contrasting color. The position of the sealing ring can be determined by means of a rod-shaped optical sensor strip 106 which is arranged on the outside of the device.

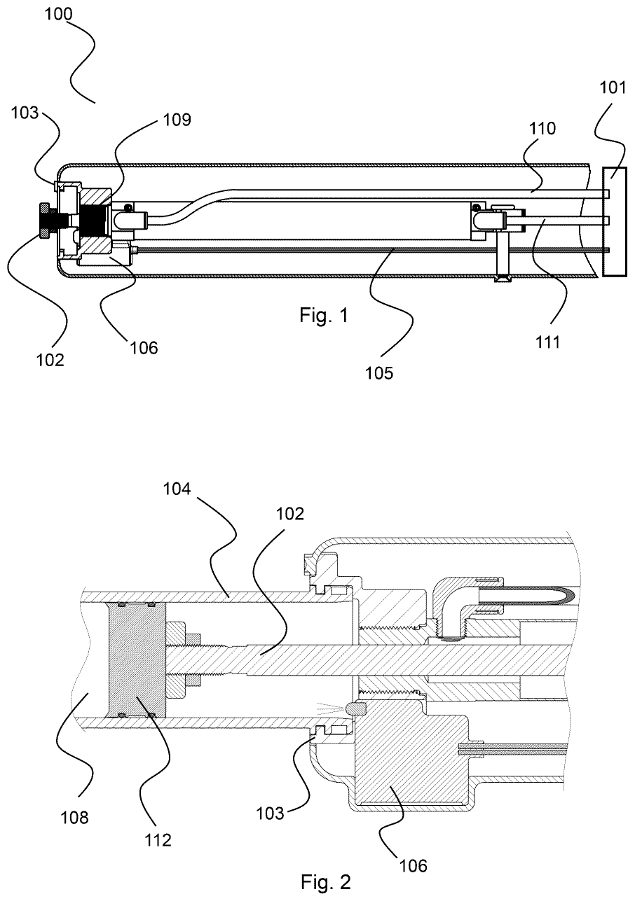

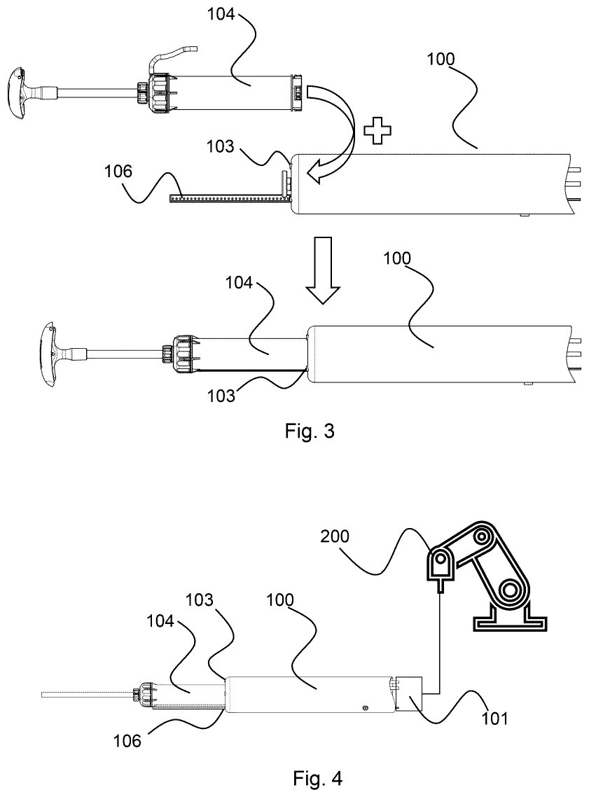

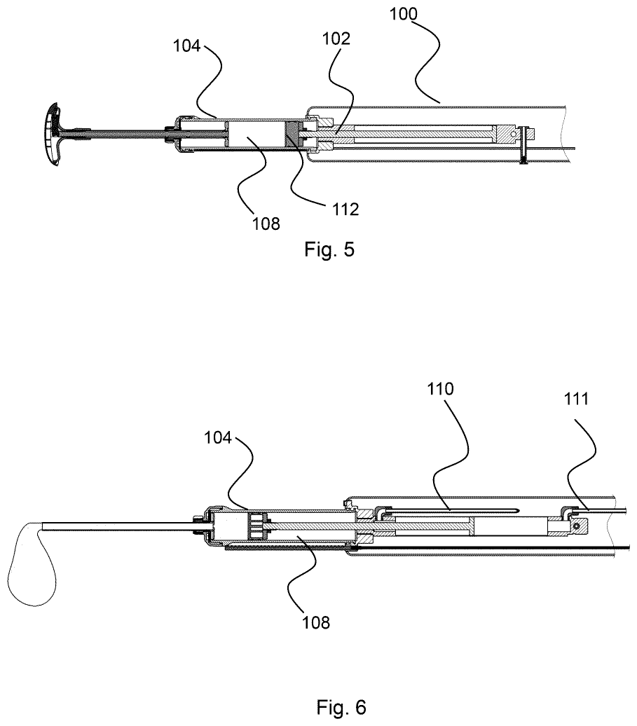

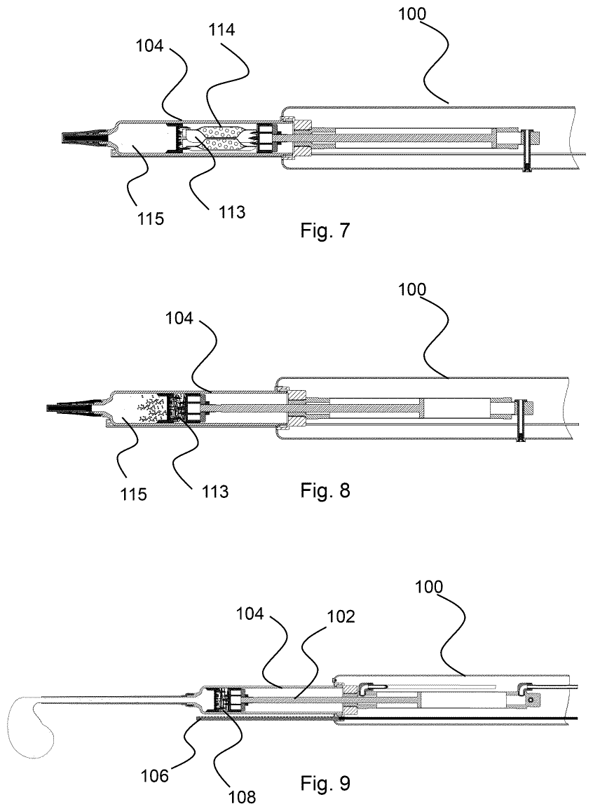

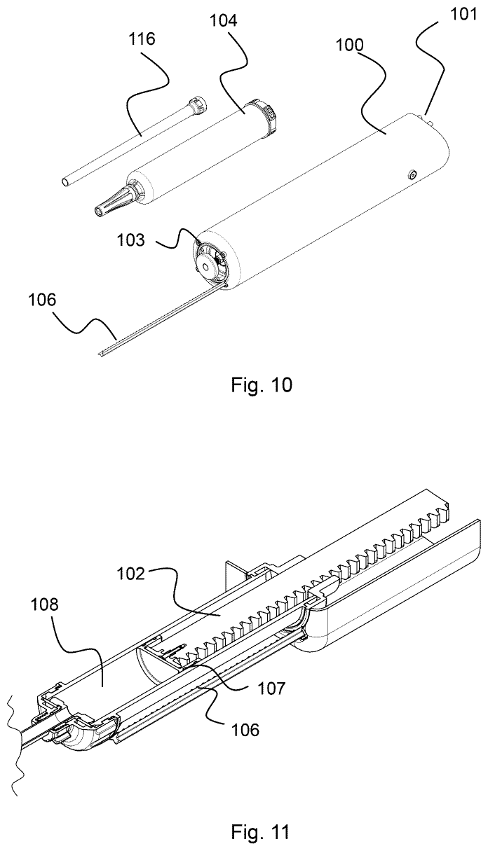

### LIST OF REFERENCE NUMERALS

100 Device
101 Connection unit
102 Press-out unit
103 Receiving unit
104 Container
105 Interface
106 Sensor
107 Marking
108 Inner volume
109 Drive
110 Inlet
111 Outlet
112 Plate
113 Chamber for liquid component
114 Bag
115 Chamber for powder component
116 Nozzle
117 Handle
200 Surgical robot

1. A device for dispensing bone cement by means of a machine, comprising a connection unit configured for connection to a surgical robot, a press-out unit configured for dispensing bone cement, a receiving unit configured for receiving a container containing bone cement, and an interface configured for connection to a control unit, in order to dispense a predefined amount of bone cement from the container by means of the press-out unit.

2. The device according to claim 1, wherein the interface is configured to convey the amount of bone cement dispensed by means of the press-out unit and to stop further dispensing of the bone cement when the predefined amount is reached.

3. The device according to claim 1, also comprising a sensor which is operatively connected to the interface in order to determine the amount of bone cement dispensed by means of the press-out unit.

4. The device according to claim 3, wherein the sensor is an optical sensor, a mechanical sensor or a magnetic sensor.

5. The device according to claim 3, wherein the sensor is configured to detect a spatial position of a marking in order to thereby determine the amount of bone cement dispensed by means of the press-out unit, wherein the marking is preferably a colored component of the press-out unit, particularly preferably a sealing ring of a plunger.

6. The device according to claim 3, wherein the sensor and/or the interface is/are configured to determine an inner volume of the press-out unit and/or of the container.

7. The device according to claim 3, wherein the sensor is arranged on a sensor strip which extends along the receiving unit on the outside of the device.

8. The device according to claim 1, wherein the connection unit comprises an electrical connection point which is configured to supply power to the device and/or to receive a control signal.

9. The device according to claim 1, wherein the connection unit comprises a connection point for connection to a pressurized medium, wherein the pressurized medium is preferably compressed air, a vacuum source or a hydraulic medium.

10. The device according to claim 1, wherein the press-out unit comprises a drive which is configured for dispensing bone cement by means of the press-out unit.

11. The device according to claim 10, wherein the drive is a hydraulic, pneumatic or electrical drive.

12. The device according to claim 10, wherein the interface is configured to control the drive.

13. The device according to claim 1, wherein the device is also configured for mixing bone cement by means of a machine in the container.

14. A computer program for controlling the device according to claim 1, wherein the computer program is configured to operate the press-out unit and/or the sensor.

15. A kit comprising a device according to claim 1 and a container containing bone cement, wherein the container is preferably configured for mixing bone cement.
